(19) United States
(12) Patent Application Publication
(10) Pub. No.: US 2023/0310496 A1
(43) Pub. Date: Oct. 5, 2023

(54) COMPOSITIONS AND METHODS FOR PREVENTING AND/OR AMELIORATING ORGAN-SPECIFIC AUTOIMMUNE DISEASES

(71) Applicant: MiZ Company Limited, Kamakura-shi (JP)

(72) Inventors: Yusuke ICHIKAWA, Kamakura-shi (JP); Fumitake SATOH, Kanagawa (JP); Bunpei SATO, Kamakura-shi (JP); Yasuko GINJYO, Kamakura-shi (JP)

(73) Assignee: MiZ Company Limited, Kamakura-shi (JP)

(21) Appl. No.: 18/185,689

(22) Filed: Mar. 17, 2023

(30) Foreign Application Priority Data
Mar. 23, 2022 (JP) JP 2022-64782
Jan. 20, 2023 (JP) JP 2023-17175

(51) Int. Cl. A61K 33/00; A61K 9/00; A61P 37/06

(52) U.S. Cl. CPC A61K 33/00; A61K 9/007; A61P 37/06

(57) ABSTRACT

Provided is a composition for improving symptoms of organ-specific autoimmune diseases. A composition comprising a hydrogen gas-containing gas as an active ingredient, for use in improving symptoms caused by an organ-specific autoimmune disease and/or reducing worsening of the symptoms in patients with an organ-specific autoimmune disease, and a method for improving symptoms including a decrease in activity levels involved in symptoms of an organ-specific autoimmune disease and/or reducing worsening of the symptoms by administering the composition to patients with the organ-specific autoimmune disease.

### RELATED APPLICATIONS

The present patent document claims the benefit of priority to Japanese Patent Application No. JP 2022-64782, filed Mar. 23, 2022, and Japanese Patent Application No. 2023-17175, filed Jan. 20, 2023, the entire contents of which are incorporated herein by reference.

### BACKGROUND

### 1. Field of the Invention

Described herein is a composition for preventing and/or improving organ-specific autoimmune diseases. Also, described herein is a method for preventing and/or improving organ-specific autoimmune diseases.

### 2. Description of the Related Art

Organ-specific autoimmune diseases are autoimmune diseases that are limitedly found in particular organs and include autoimmune hemolytic anemia, thrombocytopenic purpura, Hashimoto’s disease, Guillain-Barre syndrome, Basedow’s disease, Type I diabetes, myasthenia gravis, autoimmune gastritis and pemphigus.

In recent years, it has been proposed that hydrogen molecules may be effective against many diseases caused by chronic inflammations by scavenging hydroxyl radicals generated inside cells or mitochondria of cells, thereby reducing chronic inflammations (Shin-ichi Hirano, Yusuke Ichikawa, Bunpei Sato, Haru Yamamoto, Yoshiyasu Takefuji, Fumitake Satoh “Potential Therapeutic Applications of Hydrogen in Chronic Inflammatory Diseases: Possible Inhibiting Role on Mitochondrial Stress” International Journal of Molecular Science, 2021, 22, 2549). However, there have been no reports on whether a hydrogen gas-containing gas, which was inhaled, suctioned or drunk by patients with organ-specific autoimmune diseases, prevented and/or improved the organ-specific autoimmune diseases.

An object of the present invention is to provide a novel composition that has few side effects and can be easily produced, for use in preventing and/or improving organ-specific autoimmune diseases.

### SUMMARY

As a result of extensive research, the present inventors unexpectedly found that a hydrogen gas-containing gas prevents and/or improves organ-specific autoimmune diseases.

Therefore, described herein are the following embodiments.

(1) A composition comprising hydrogen as an active ingredient, for use in improving symptoms caused by an organ-specific autoimmune disease and/or reducing worsening of the symptoms in a subject.

(2) The composition according to (1), wherein the organ-specific autoimmune disease is selected from one or more diseases consisting of myasthenia gravis, Hashimoto’s disease, Basedow’s disease, ulcerative colitis, autoimmune hemolytic anemia, thrombocytopenic purpura, Guillain-Barre syndrome, type I diabetes, autoimmune gastritis and pemphigus.

(3) The composition according to (1) or (2), wherein the symptoms caused by an organ-specific autoimmune disease include: symptoms in myasthenia gravis, such as systemic muscle weakness, limb muscle weakness, fatigability, ptosis, eye symptoms such as double vision, dysphagia, dyspnea and a decrease in quality of life associated with these symptoms, and side effects of myasthenia gravis therapeutic drugs; symptoms in Hashimoto’s disease, such as fatigue, coldness, body swelling, weight gain, drowsiness, memory loss, shortness of breath, decreased pulse rate, muscle weakness, bradykinesia, anovulation, amenorrhea, constipation, thyroid gland swelling, eyelid swelling, depression, constipation, hypercholesterolemia and a decrease in quality of life associated with these symptoms, and side effects of Hashimoto’s disease therapeutic drugs; symptoms in Basedow’s disease, such as fatigue, excessive sweating, hot flashes, weight loss, arrhythmia, hand tremors, nervousness, anxiety, irritability, sleep disorder, increased number of claustrophobia, decreased cholinesterase, oligomenorrhea, amenorrhea, loose stools, thyroid gland swelling, ocular proptosis and a decrease in quality of life associated with these symptoms, and side effects of Basedow’s disease therapeutic drugs; symptoms in ulcerative colitis, such as hematochezia, viscous stool, diarrhea, bloody diarrhea, abdominal pain, fever, anorexia, weight loss, anemia, arthritis, iritis, pancreatitis, erythema nodosum, pyoderma gangrenosum and a decrease in quality of life associated with these symptoms, and side effects of ulcerative colitis therapeutic drugs; symptoms in autoimmune hemolytic anemia, such as anemia-related lethargy, anemia-related palpitations, anemia-related shortness of breath, anemia-related dizziness, anemia-related headaches, jaundice, spleen swelling and a decrease in quality of life associated with these symptoms, and side effects of autoimmune hemolytic anemia therapeutic drugs; symptoms in thrombocytopenic purpura, such as gingival bleeding, epistaxis, subcutaneous bleeding, genital bleeding, hematochezia and a decrease in quality of life associated with these symptoms, and side effects of thrombocytopenic purpura therapeutic drugs; and symptoms in Guillain-Barre syndrome, such as muscle weakness, dysesthesia, anesthesia, pain, ataxia, autonomic neuropathy and a decrease in quality of life associated with these symptoms, and side effects of Guillain-Barre syndrome therapeutic drugs; and symptoms associated with an organ-specific autoimmune disease include: symptoms in type I diabetes, such as thirst, frequent urination, rapid weight loss, fatigue and a decrease in quality of life associated with these symptoms, and side effects of type I diabetes therapeutic drugs; symptoms in autoimmune gastritis, such as gastric atrophy, pernicious anemia, numbness of the fingertips, gait disturbance and a decrease in quality of life associated with these symptoms, and side effects of autoimmune gastritis therapeutic drugs; and symptoms in pemphigus, such as oral mucosal erosions, oral mucosal ulcers, cutaneous pemphigus foliaceus and a decrease in quality of life associated with these symptoms, and side effects of pemphigus therapeutic drugs.

(4) The composition according to any one of (1) to (3), wherein the concentration of hydrogen is more than 0 and 18.5 volume % or less.

(5) The composition according to any one of (1) to (4), wherein the composition is administered to the subject by inhalation.

(6) The composition according to any one of (1) to (5), wherein the subject is a mammal including a human.

(7) A method for producing the composition according to any one of (1) to (6), comprising using a hydrogen gas generator, thereby producing the composition.

### Effects

The presently described composition and method make it possible to prevent and/or improve organ-specific autoimmune diseases by hydrogen gas.

### DETAILED DESCRIPTION OF THE PREFERRED EMBODIMENTS

The present compositions and methods will be described in more detail below.

### 1. Organ-Specific Autoimmune Disease

Examples of the organ-specific autoimmune diseases include myasthenia gravis, Hashimoto’s disease, Basedow’s disease, ulcerative colitis, autoimmune hemolytic anemia, thrombocytopenic purpura, Guillain-Barre syndrome, type I diabetes, autoimmune gastritis and pemphigus.

### 2. Composition Containing a Hydrogen Gas-Containing Gas for Use in Preventing and/or Improving Organ-Specific Autoimmune Diseases

Described herein is a composition containing a hydrogen gas-containing gas as an active ingredient, for use in improving symptoms caused by organ-specific autoimmune diseases and/or reducing worsening of the symptoms.

Examples of the symptoms associated with organ-specific autoimmune diseases include symptoms in myasthenia gravis, such as systemic muscle weakness, limb muscle weakness, fatigability, ptosis, eye symptoms such as double vision, dysphagia, dyspnea and a decrease in quality of life associated with these symptoms, and side effects of myasthenia gravis therapeutic drugs.

Recently, particularly in old people, the number of patients with myasthenia gravis, has increased. Since most old people have a weakened immune system due to, e.g., thymoma, different from young people, steroid drugs are used for treatment. However, treatment with steroid drugs imposes a heavy burden on the old people. Steroid drugs also have side effects such as cataract and osteoporosis. Then, an immunosuppressant is used but it involves risks of developing infections and cancers. Most of the old people with myasthenia gravis has a prognosis: die of cancer within 10 years. Then, it is expected that onset of cancers caused by drugs of myasthenia gravis is prevented by hydrogen.

Examples of the symptoms associated with organ-specific autoimmune diseases include symptoms in Hashimoto’s disease, such as fatigue, coldness, body swelling, weight gain, drowsiness, memory loss, shortness of breath, decreased pulse rate, muscle weakness, bradykinesia, anovulation, amenorrhea, constipation, thyroid gland swelling, eyelid swelling, depression, constipation, hypercholesterolemia, and a decrease in quality of life associated with these symptoms, and side effects of Hashimoto’s disease therapeutic drugs.

Examples of symptoms associated with organ-specific autoimmune diseases include symptoms in Basedow’s disease, such as fatigue, excessive sweating, hot flashes, weight loss, arrhythmia, hand tremors, nervousness, anxiety, irritability, sleep disorder, increased number of claustrophobia, decreased cholinesterase, oligomenorrhea, amenorrhea, loose stools, thyroid gland swelling, ocular proptosis and a decrease in quality of life associated with these symptoms, and side effects of Basedow’s disease therapeutic drugs.

Examples of the symptoms associated with organ-specific autoimmune diseases include symptoms in ulcerative colitis, such as hematochezia, viscous stool, diarrhea, bloody diarrhea, abdominal pain, fever, anorexia, weight loss, anemia, arthritis, iritis, pancreatitis, erythema nodosum, pyoderma gangrenosum and a decrease in quality of life associated with these symptoms, and side effects of ulcerative colitis therapeutic drugs.

Examples of the symptoms associated with organ-specific autoimmune diseases include symptoms in autoimmune hemolytic anemia, such as anemia-related lethargy, anemia-related palpitations, anemia-related shortness of breath, anemia-related dizziness, anemia-related headaches, jaundice, spleen swelling and a decrease in quality of life associated with these symptoms, and side effects of autoimmune hemolytic anemia therapeutic drugs.

Examples of the symptoms associated with organ-specific autoimmune diseases include symptoms in thrombocytopenic purpura, such as gingival bleeding, epistaxis, subcutaneous bleeding, genital bleeding, hematochezia, a decrease in quality of life associated with these symptoms, and side effects of thrombocytopenic purpura therapeutic drugs.

Examples of the symptoms associated with organ-specific autoimmune diseases include symptoms in Guillain-Barre syndrome, such as muscle weakness, dysesthesia, anesthesia, pain, ataxia, autonomic neuropathy and a decrease in quality of life associated with these symptoms, and side effects of Guillain-Barre syndrome therapeutic drugs.

Examples of the symptoms associated with organ-specific autoimmune diseases include symptoms in type I diabetes, such as thirst, frequent urination, rapid weight loss, fatigue and a decrease in quality of life associated with these symptoms, and side effects of type I diabetes therapeutic drugs.

Examples of the symptoms associated with organ-specific autoimmune diseases include symptoms in autoimmune gastritis, such as gastric atrophy, pernicious anemia, numbness of the fingertips, gait disturbance and a decrease in quality of life associated with these symptoms, and side effects of autoimmune gastritis therapeutic drugs.

Examples of the symptoms associated with organ-specific autoimmune diseases include symptoms in pemphigus, such as oral mucosal erosions, oral mucosal ulcers, cutaneous pemphigus foliaceus and a decrease in quality of life associated with these symptoms, and side effects of pemphigus therapeutic drugs.

In the present specification, “hydrogen,” which is an active ingredient of the described composition, is molecular hydrogen (i.e., gaseous hydrogen), and is simply referred to as “hydrogen” or “hydrogen gas” unless otherwise specified. Further, the term “hydrogen” used in the present specification refers to hydrogen represented by the molecular formula of H₂, D₂ (deuterium), or HD (hydrogen deuteride), or a gas mixture thereof. D₂ is expensive, but is known to have a stronger superoxide scavenging effect than H₂. The type of hydrogen usable in the compositions and methods described herein is H₂, D₂ (deuterium), HD (hydrogen deuteride), or a gas mixture thereof, and preferably H₂. Alternatively, D₂ and/or HD may be used in place of H₂, or as a mixture with H₂.

The hydrogen gas-containing gas is preferably air containing hydrogen gas, or a gas mixture containing hydrogen gas and oxygen gas. The concentration of hydrogen gas in the hydrogen gas-containing gas is more than 0 and 18.5 volume % or less, and preferably more than 0 and 10 volume % or less. Here, the effect of improving symptoms of organ-specific autoimmune diseases and/or reducing worsening of the symptoms tends to be enhanced as the hydrogen gas concentration increases at a level equal to or less than the explosion limit.

Since hydrogen is a flammable and explosive gas, in the improvement of symptoms of organ-specific autoimmune diseases and/or reducing worsening of the symptoms, it is preferable to incorporate hydrogen into the described composition under safe conditions and then administer the composition to patients with organ-specific autoimmune diseases.

When the gas other than hydrogen gas is air, the air concentration is within the range of 81.5 to 99.5 volume %, for example. When the gas other than hydrogen gas is a gas containing oxygen gas, the concentration of oxygen gas is within the range of 21 to 99.5 volume %, for example.

Nitrogen gas can be contained as another main gas. A gas, such as carbon dioxide, which is a gas present in the air, may be contained in an amount equivalent to the abundance thereof in the air.

As described herein, a hydrogen-dissolved liquid can be administered to or ingested by a patient with organ-specific autoimmune diseases in combination with the administration of the hydrogen gas-containing gas, as necessary.

In the case of combined administration with a hydrogen-dissolved liquid, the described composition can be administered before the administration of the hydrogen-dissolved liquid, simultaneously with the administration of the hydrogen-dissolved liquid, or after the administration of the hydrogen-dissolved liquid.

The hydrogen-dissolved liquid is specifically an aqueous liquid in which hydrogen gas is dissolved. Non-limitative examples of the aqueous liquid include water (e.g., sterile water and purified water), saline, buffer solutions (e.g., buffer solutions having a pH of 4 to 7.4), ethanol-containing water (e.g., ethanol content: 0.1 to 2 volume %), intravenous drips, infusion solutions, injection solutions, beverages, and the like. The hydrogen concentration of the hydrogen-dissolved liquid is, for example, 1 to 10 ppm, for example, 2 to 8 volume %, 3 to 7 volume %, 3 to 6 volume %, 4 to 6 volume %, 4 to 5 volume %, 5 to 10 volume %, 5 to 8 volume %, 6 to 8 volume %, or 6 to 7 volume %, and more preferably 5 to 8 volume %, for example, 6 to 8 volume %, or 6 to 7 volume %. As described herein, the effect of improving symptoms of organ-specific autoimmune diseases and/or reducing worsening of the symptoms tends to be enhanced as the hydrogen gas concentration increases at a level equal to or less than the explosion limit.

A drug for treating organ-specific autoimmune diseases may be added to the hydrogen-dissolved liquid. Alternatively, such a drug may be administered separately from the administration of the hydrogen-dissolved liquid or hydrogen gas-containing gas.

The hydrogen gas-containing gas or hydrogen-dissolved liquid is formulated to a predetermined hydrogen gas concentration, and then placed in, for example, a pressure-resistant container (e.g., a stainless-steel cylinder or an aluminum can, preferably a pressure-resistant plastic bottle laminated with an aluminum film on the inside (e.g., a pressure-resistant PET bottle), a plastic bag, an aluminum bag, etc.). Aluminum has the property of being impermeable to hydrogen molecules. Alternatively, the hydrogen gas-containing gas or hydrogen-dissolved liquid may be produced in situ at the time of administration using a device, such as a hydrogen gas generator, a hydrogen water generator, or a hydrogen gas-adding device, for example, a known or commercially available hydrogen gas supply device (a device for generating a hydrogen gas-containing gas),a hydrogen-adding instrument (a device for generating hydrogen water, or a non-destructive hydrogen-adding apparatus (e.g., a device for non-destructively adding hydrogen gas into a bag for a biologically applicable liquid, such as an intravenous drip).

The hydrogen gas supply device makes it possible to mix hydrogen gas generated by the reaction of a hydrogen-generating agent (e.g., metallic aluminum, magnesium hydride, etc.) and water, with a diluting gas (e.g., air, oxygen, etc.) at a predetermined ratio (Japanese Patent No. 5228142, etc.). Alternatively, hydrogen gas generated by using electrolysis of water is mixed with a diluting gas, such as oxygen or air (Japanese Patent No. 5502973, Japanese Patent No. 5900688, etc.). In this way, a hydrogen gas-containing gas having a hydrogen concentration within the range of 0.5 to 18.5 volume % can be prepared.

The hydrogen-adding instrument is a device that generates hydrogen using a hydrogen-generating agent and a pH adjusting agent, and dissolves it in a biologically applicable liquid, such as water (Japanese Patent No. 4756102, Japanese Patent No. 4652479, Japanese Patent No. 4950352, Japanese Patent No. 6159462, Japanese Patent No. 6170605, Japanese Patent Laid-Open No. 2017-104842, Japanese Patent No. 6159462, etc.). The combination of a hydrogen-generating agent and a pH adjusting agent is, for example, a combination of metallic magnesium and a strongly acidic ion-exchange resin or an organic acid (e.g., malic acid, citric acid, etc.), a combination of metallic aluminum powder and calcium hydroxide powder, and the like. In this way, a hydrogen-dissolved liquid with a dissolved hydrogen concentration of about 1 to 10 ppm can be prepared (e.g., trade name “7 Water” (MiZ Company Limited), etc.).

The non-destructive hydrogen-adding apparatus is a device or instrument that adds hydrogen molecules to a commercially available biologically applicable liquid (e.g., enclosed in a hydrogen-permeable plastic bag, such as a polyethylene bag), such as an intravenous drip, from the outside of the package, and is commercially available from, for example, MiZ Company Limited (http://www.e-miz.co.jp/technology.html). With this apparatus, a bag containing a biologically applicable liquid is immersed in saturated hydrogen water so that the bag is permeated with hydrogen, whereby hydrogen can be aseptically dissolved in the biologically applicable liquid until reaching concentration equilibrium. The apparatus includes, for example, an electrolyzer and a water tank, and water in the water tank circulates between the electrolyzer and the water tank, and can generate hydrogen by electrolysis. Alternatively, a simple disposable instrument can be used for the same purpose (Japanese Patent Laid-Open No. 2016-112562, etc.). This instrument includes a biologically applicable liquid-containing plastic bag (a hydrogen-permeable bag, such as a polyethylene bag) and a hydrogen-generating agent (e.g., metallic calcium, metallic magnesium/cation-exchange resin, etc.) in an aluminum bag, and the hydrogen-generating agent is covered with, for example, a non-woven fabric (e.g., a water vapor-permeable non-woven fabric). Hydrogen generated by wetting the hydrogen-generating agent covered with a non-woven fabric with a small amount of water, such as water vapor, passes through the plastic bag and is non-destructively and aseptically dissolved in the biologically applicable liquid.

The hydrogen gas-containing gas or hydrogen saturated biologically applicable liquid (e.g., sterile water, saline, an intravenous drip, etc.) prepared using the device or instrument described above can be administered orally or parenterally to a patient with organ-specific autoimmune diseases.

In another embodiment, the described composition includes dosage forms (e.g., tablets, capsules, etc.) containing a hydrogen-generating agent that can generate hydrogen in the gastrointestinal tract, prepared to be orally administered to (or ingested by) a patient with organ-specific autoimmune diseases. The hydrogen-generating agent is preferably composed of, for example, ingredients approved as foods or food additives.

### 3. Improvement of Symptoms of Organ-Specific Autoimmune Diseases and/or Suppression of Worsening of Symptoms

In a second embodiment, described is a method for improving symptoms, which are involved in the symptoms caused by organ-specific autoimmune diseases and includes decrease in activity levels, and/or reducing worsening of the symptoms, in patients with organ-specific autoimmune diseases, the method comprising administering the described composition to the patients.

The described composition can improve the quality of life (QOL) of patients.

As the method for administering the described composition to a patient with organ-specific autoimmune diseases, when hydrogen gas is used as an active ingredient, for example, pulmonary administration by inhalation, sucking, or the like is preferred. When the gas is inhaled, the gas can be inhaled from the mouth or nose through a nasal cannula or a mask-like instrument that covers the mouth and nose, and the gas can be delivered to the lungs and distributed throughout the body via blood.

When a hydrogen-dissolved liquid is administered to a patient, oral administration, intravenous administration, or intraarterial administration (including infusion) is preferred. The hydrogen-dissolved liquid to be orally administered is preferably stored at low temperatures, and the cooled liquid or the liquid stored at ordinary temperature may be administered to a patient with organ-specific autoimmune diseases. Hydrogen is dissolved in water at a concentration of about 1.6 ppm (1.6 mg/L) at ordinary temperature and pressure, and it is known that the difference in solubility due to temperature is relatively small. Alternatively, when the hydrogen-dissolved liquid is in the form of, for example, a hydrogen gas-containing intravenous drip or injection prepared using the non-destructive hydrogen-adding apparatus mentioned above, it may be administered to a patient with organ-specific autoimmune diseases by a parenteral administration route, such as intravenous administration or intraarterial administration.

A hydrogen gas-containing gas with the above hydrogen concentration or a hydrogen-dissolved liquid with the above dissolved hydrogen concentration can be administered to a patient with organ-specific autoimmune diseases once or several times (e.g., 2 or 3 times) a day over 1 week to 3 months or a longer period of time, for example, 1 week to 6 months or longer. When a hydrogen gas-containing gas is administered it can be administered per dose over for example, 10 minutes to 2 hours or longer, preferably 20 minutes to 40 minutes or longer, and more preferably 30 minutes to 2 hours. Further, when a hydrogen gas-containing gas is pulmonary administered by inhalation, sucking, or the like, the gas can be administered to a patient with organ-specific autoimmune diseases in an atmospheric pressure environment or, for example, at a high pressure within the range of more than the normal atmospheric pressure (about 1.013 atm) and 7.0 atm or less, for example, in a high-pressure environment within the range of 1.02 to 7.0 atm, preferably 1.02 to 5.0 atm, more preferably 1.02 to 4.0 atm, and further preferably 1.02 to 1.35 atm. Administration in a high-pressure environment can promote the systemic absorption of hydrogen in the patient with organ-specific autoimmune diseases.

The high-pressure environment can be created by using a high-pressure housing (e.g., a capsule-like housing) that is designed to have sufficient strength and that can form a high pressure of more than the normal atmospheric pressure and 7.0 atm or less inside, for example, by pressing the hydrogen gas-containing gas (e.g., hydrogen-containing oxygen or air) thereinto. The shape of the high-pressure housing is preferably rounded overall with no corners because it is pressure resistant. Further, the material of the high-pressure housing is preferably lightweight and high-strength, and examples include reinforced plastics, carbon fiber composite materials, titanium alloys, aluminum alloys, and the like. Patients with organ-specific autoimmune diseases can undergo administration of a composition containing hydrogen gas together with oxygen gas or air, for use in improving symptoms of organ-specific autoimmune diseases and/or reducing worsening of the symptoms, in such a high-pressure housing.

In the treatment of organ-specific autoimmune diseases using the described composition, it is desirable to use a hydrogen gas generator, a hydrogen water generator, or a hydrogen gas-adding device (e.g., the above-mentioned hydrogen gas supply device (or a gaseous hydrogen inhalation device), a hydrogen-adding instrument (or a hydrogen water generator), a non-destructive hydrogen-adding apparatus (a device that non-destructively dissolves hydrogen gas in a biologicallyapplicable liquid, such as an intravenous drip enclosed in a hydrogen-permeable bag), and other devices), for which sufficient therapeutic efficacy and safety have been confirmed.

### EXAMPLES

Preferred embodiments will be described in more detail below with reference to Examples; however, the technical scope of the described compositions and methods is not limited to these Examples.

### [Example 1] Improvement of Symptoms of Myasthenia Gravis and/or Suppression of Worsening of Symptoms by Hydrogen Gas Inhalation

### (Course of Disease Before Inhalation of Hydrogen Gas)

Patient: Japanese woman aged 64 at the time of onset. The course from onset is as follows:

Aug. 22, 2021: symptom of double vision suddenly occurred.

Aug. 24, 2021: symptom of ptosis occurred.

Aug. 26, 2021: upper lip paralysis occurred. The patient consulted a doctor of the Department of Neurology in the Juntendo University Hospital. Since the patient was positive on an ice pack test and a Tensilon test, she was diagnosed as myasthenia gravis. No abnormality was found in a CT test. As a result of a blood test, the patient was false positive to an anti-acetylcholine antibody and prescribed an immunosuppressant (Tacrolimus 3 mg/day). Although the patient started taking of the immunosuppressant, the symptoms progressed. The patient could not read letters on a newspaper, raise a frying pan or write letters, and her chewing power reduced.

Sep. 9, 2021: the patient consulted the doctor because of worsening of symptoms and prescribed an anticholinesterase agent (Mestinon 60 mg × 3/day). However, her symptoms further got worse. Every day after 3 p.m. or later, the patient felt that her arms are unbearably heavy. Since the arms cannot be removed, the patient spent time with the arms kept on a table.

Sep. 25, 2021: since symptoms were not improved and merely got worse, the patient changed her primary doctor to a neuroimmunology specialist doctor and hospitalization was considered.

Sep. 29, 2021: an immunosuppressant (1 mg) was additionally prescribed by the neuroimmunology specialist doctor; that is, immunosuppressants (Tacrolimus 3 mg/day, Prograf 1 mg/day) were prescribed. The patient avoided hospitalization but was followed up as an outpatient. As a result of a blood test performed later, it was found that the patient was positive to an anti-MuSK antibody. Based on the result, the patient was definitively diagnosed as anti-MuSK antibody positive myasthenia gravis.

Nov. 10, 2022: when the patient drank beer only a little, her face turned red, although the patient was able to drink alcohol by nature. This was considered probably due to side effects of the drugs.

Jan. 5, 2022: although progression of the symptoms was stopped by administration of the drugs, muscle weakness still persisted without improvement. As a result of a blood test on November 10, the number of red blood cells and hematocrit decreased to standard values or less by side effects of the administration drugs. The changes in the number of red blood cells and hematocrit from September 29 to November 10 are as follows:
Number of red blood cells: 4.64 (10¹²/L) → 3.75 (10¹²/L),
Hematocrit: 44.0 (%) → 35.3 (%).
Normal values of the number of red blood cells and hematocrit are 3.8 to 5.04 (10¹²/L) and 35.6 to 45.4 (%), respectively.

### (Course of Disease After Inhalation of Hydrogen Gas)

Jan. 23, 2022: use of the drugs of myasthenia gravity has a risk of producing side effects, such as cancer and osteoporosis. Thus, in order to reduce the amounts of the drugs and improve symptoms, the patient started inhalation of hydrogen gas. The hydrogen-gas inhaler used herein was Jobs-α (trade name, registered as a trademark as Jobs) manufactured by MiZ Company Limited. (Kamakura city, Kanagawa prefecture, Japan). The concentration of hydrogen provided by Jobs-α is about 4% to 5%, the full (100%) generation capacity of hydrogen is 200 ml/min.

Jan. 28, 2022: Day 5 after initiation of the inhalation, languor of the arm was completely gone.

Feb. 1, 2022: constipation, which was a side effect of the immunosuppressants, was overcome and the patient felt good. On March 9, the day the patient was scheduled to visit the hospital, she started to think that she would like to ask the doctor to reduce the amount of a drug. After inhalation of hydrogen, even if she drank alcohol, her face never turned red. The patient was quite sure that side effects of the drugs are suppressed.

Feb. 16, 2022: the patient was aware that blemishes on her face became light and felt that physical conditions are recovering by hydrogen inhalation.

Mar. 9, 2022: as a result of a blood test, the number of red blood cells and hematocrit were, 4.22 (10¹²/L) and 40.7 (%), respectively. Since side effects of myasthenia gravis therapeutic drugs were reduced by hydrogen inhalation, she daringly stopped administration of immunosuppressants while monitoring her physical conditions.

Mar. 20, 2022: symptoms of myasthenia gravis were no longer observed by inhalation of hydrogen gas.

Mar. 22, 2022: when inhaled hydrogen gas, she felt that the body gets lighter and became to enjoy walking.

Mar. 30, 2022: usually when use of an immunosuppressant is stopped, relapse occurs at a high probability. However, the patient who was sure that the effect provided by inhalation of hydrogen gas is larger than that provided by the immunosuppressants, stopped use of the immunosuppressants and continued only inhalation of hydrogen gas.

Jun. 30, 2022: since use of the immunosuppressants was stopped, no relapse has taken place. This fact surprised the doctor. The patient was sure that hydrogen has a prevention and improvement effect on myasthenia gravis. The patient supplementally drank hydrogen water once every 2 to 4 days (7 WATER manufactured by MiZ Company Limited.; hydrogen concentration: 7 ppm, 500 ml).

Aug. 24, 2022: medical examination was carried out at the Department of Neurology in the Juntendo University Hospital. As a result, the patient was still negative to an anti-MuSK antibody and no relapse occurred.

Oct. 19, 2022: the patient was told by a doctor of the Department of Neurology in the Juntendo University Hospital that myasthenia gravis was not in remission but in healed state. Since then, the patient has needed not to see a doctor.

### [Example 2] Improvement of Symptoms of Hashimoto’s Disease and/or Suppression of Worsening of Symptoms by Inhalation of Hydrogen Gas

Patient: Japanese man aged 50. When the patient was diagnosed as Basedow’s disease by a doctor and took an antithyroid drug, secretion of thyroid hormone decreased due to a side effect of the drug. The patient developed Hashimoto’s disease. The symptoms of Hashimoto’s disease were fatigue and dark visual field. When inhalation of hydrogen gas was initiated by use of MHG-2000α (trade name) (concentration of hydrogen: about 5% to 7%, the full (100%) generation capacity of hydrogen: 120 ml/min) manufactured by MiZ Company Limited. (Kamakura city, Kanagawa prefecture, Japan), the patient felt less fatigued and the visual field was recovered from darkness and became bright.

### INDUSTRIAL APPLICATION

The described compositions and methods make it possible to improve symptoms of organ-specific autoimmune diseases and/or symptoms such as decrease in activity levels involved in the symptoms of the diseases and/or to reduce worsening of the symptoms just by administering hydrogen to patients with organ-specific autoimmune diseases. Since side effects of hydrogen itself are not found, the QOL of the patients can be enhanced.

What is claimed is:

1. A method for preventing and improving symptoms caused by an organ-specific autoimmune disease and a side effect of the organ-specific autoimmune disease and/or reducing worsening of the symptoms in a subject, comprising administering to the subject a composition comprising hydrogen as an active ingredient.

2. The method according to claim 1, wherein the organ-specific autoimmune disease is selected from one or more diseases consisting of myasthenia gravis, Hashimoto’s disease, Basedow’s disease, ulcerative colitis, autoimmune hemolytic anemia, thrombocytopenic purpura, Guillain-Barre syndrome, type I diabetes, autoimmune gastritis and pemphigus.

3. The method according to claim 1, wherein the symptoms caused by an organ-specific autoimmune disease include:
symptoms in myasthenia gravis, such as systemic muscle weakness, limb muscle weakness, fatigability, ptosis, eye symptoms such as double vision, dysphagia, dyspnea and a decrease in quality of life associated with these symptoms, and side effects of myasthenia gravis therapeutic drugs;
symptoms in Hashimoto’s disease, such as fatigue, coldness, body swelling, weight gain, drowsiness, memory loss, shortness of breath, decreased pulse rate, muscle weakness, bradykinesia, anovulation, amenorrhea, constipation, thyroid gland swelling, eyelid swelling, depression, constipation, hypercholesterolemia and a decrease in quality of life associated with these symptoms, and side effects of Hashimoto’s disease therapeutic drugs;
symptoms in Basedow’s disease, such as fatigue, excessive sweating, hot flashes, weight loss, arrhythmia, hand tremors, nervousness, anxiety, irritability, sleep disorder, increased number of claustrophobia, decreased cholinesterase, oligomenorrhea, amenorrhea, loose stools, thyroid gland swelling, ocular proptosis and a decrease in quality of life associated with these symptoms, and side effects of Basedow’s disease therapeutic drugs;
symptoms in ulcerative colitis, such as hematochezia, viscous stool, diarrhea, bloody diarrhea, abdominal pain, fever, anorexia, weight loss, anemia, arthritis, iritis, pancreatitis, erythema nodosum, pyoderma gangrenosum and a decrease in quality of life associated with these symptoms, and side effects of ulcerative colitis therapeutic drugs;
symptoms in autoimmune hemolytic anemia, such as anemia-related lethargy, anemia-related palpitations, anemia-related shortness of breath, anemia-related dizziness, anemia-related headaches, jaundice, spleen swelling and a decrease in quality of life associated with these symptoms, and side effects of autoimmune hemolytic anemia therapeutic drugs;
symptoms in thrombocytopenic purpura, such as gingival bleeding, epistaxis, subcutaneous bleeding, genital bleeding, hematochezia and a decrease in quality of life associated with these symptoms, and side effects of thrombocytopenic purpura therapeutic drugs; and
symptoms in Guillain-Barre syndrome, such as muscle weakness, dysesthesia, anesthesia, pain, ataxia, autonomic neuropathy and a decrease in quality of life associated with these symptoms, and side effects of Guillain-Barre syndrome therapeutic drugs; and
symptoms associated with an organ-specific autoimmune disease include:
symptoms in type I diabetes, such as thirst, frequent urination, rapid weight loss, fatigue and a decrease in quality of life associated with these symptoms, and side effects of type I diabetes therapeutic drugs;
symptoms in autoimmune gastritis, such as gastric atrophy, pernicious anemia, numbness of the fingertips, gait disturbance and a decrease in quality of life associated with these symptoms, and side effects of autoimmune gastritis therapeutic drugs; and
symptoms in pemphigus, such as oral mucosal erosions, oral mucosal ulcers, cutaneous pemphigus foliaceus and a decrease in quality of life associated with these symptoms, and side effects of pemphigus therapeutic drugs.

4. The method according to claim 1, wherein the concentration of hydrogen is more than 0 and 18.5 volume % or less.

5. The method according to claim 1, wherein the composition is administered to the subject by inhalation.

6. The method according to claim 1, wherein the subject is a mammal including a human.

7. The method according to claim 1, wherein the composition is produced using a hydrogen gas generator.
